(19) United States
(12) Patent Application Publication
(10) Pub. No.: US 2013/0096457 A1
(43) Pub. Date: Apr. 18, 2013

(54) ORAL SCOPE SYSTEM WITH IMAGE SENSOR AND METHOD FOR VISUAL EXAMINATION OF ORAL CAVITY AND UPPER AIRWAY

(71) Applicants: Chunyuan Qiu, Huntington Beach, CA (US); Zaiqi Wang, Edison, NJ (US)

(72) Inventors: Chunyuan Qiu, Huntington Beach, CA (US); Zaiqi Wang, Edison, NJ (US)

(73) Assignee: Qscope, LLC, Huntington Beach, CA (US)

(21) Appl. No.: 13/276,267

(22) Filed: Oct. 18, 2011

(51) Int. Cl. A61B 5/01; A61B 1/012; A61B 1/24; A61B 1/04; A61B 1/005

(52) U.S. Cl. 600/549

(57) ABSTRACT

An oral scope instrument for facilitating visual examination and inspection of a person's oral cavity and upper airway includes an elongate, flexible arm having first and second ends, a temperature sensor connected to the flexible arm adjacent the first end, an image sensor mounted adjacent the second end, a processor in electrical communication with the image sensor and an image display device. An illumination unit located adjacent the second end provides illumination for the image sensor. A handle connected to the first end enables manual insertion of the flexible arm into the mouth along a portion of the top of the tongue to engage the temperature sensor therewith and position the image sensor for viewing and recording anatomical features of the oral cavity or the upper airway. The processor stores normal anatomical images and may transmit images of recorded anatomical features to the image display device.

### TECHNICAL FIELD AND BACKGROUND

Humans have the innate ability to know their abnormal feelings (i.e. symptoms) and to locate their abnormal findings (i.e signs). Because of this unique survival skill, a person is often the first to know if he or she needs to seek medical attention, and a doctor is often the first to use the symptoms and signs to make correct diagnosis. Needless to say, self-recognition of body abnormalities plays an essential role for maintaining one's health as well as battling against diseases. It is indeed the first line of human defense against wide range of diseases.

Human eyes are capable of recognizing gross abnormalities, such as lumps and bumps, skin discoloration and swelling. Thus, diagnosis based on visual examination becomes a routine procedure for some health problems. For example, in traditional Chinese medicine and modern western medicine, the examination of a patient's tongue, gums, throat and upper airways is a routine medical procedure. This procedure is also commonly done at home by a person using a flashlight and mirror, either on themself or another in the family. For example, at home, parents of small children often use teaspoons, flashlights and a mirror to look for infected spots. However, thorough inspection and examination of the oral cavity and upper airway are difficult even in the hand of experienced professionals, where a wood or metal tongue depressor is often used. This is because clearly being able to see the mouth and throat is hampered and restricted by insufficient lighting, angles of the tongue depressor and/or deeper location of infection. Most importantly, noxious stimulation (gag reflex) by a tongue depressor may make the visual examination impossible, especially in the situation where mouth and throat pain from infection and inflammation severely limit adequate opening of the mouth. This is especially true in pediatrics, where an upper airway infection such as streptococcus throat is common and children are hardly cooperative when they are sick, making it very difficult for a physician to visualize the throat and tonsil. Furthermore, the direct view through the naked eye simply cannot overcome the curvature of a normal airway to see the deeper tissue and the traditional examination with a tongue blade, even if done correctly, relies on visualization, memory and recollection of the person doing the examination, and that can vary significantly from person to person, and from time to time. Furthermore, self-examination and inspection of one's own mouth and throat is even more difficult or if not impossible in some situations.

Correct diagnosis and/or initial symptom identification both rely on accurate visual examination of the anatomy of the mouth to determine presence of a sore throat, location of a toothache, fever or other clinical problems that affect millions of people. Misdiagnosis often occurs by using a traditional tongue blade, because of the visual difficulty described above. Consequently, correct treatments cannot be established, which result in continued problems, leading to serious complication such as rheumatic heart diseases caused by streptococcal throat. On other hand, the widespread misuse/overuse of antibiotic drugs worldwide is due to incorrect diagnoses, and has contributed to the rise of multiple antibiotic resistance bacteria that endanger populations as a whole. Self-administered antibiotic use by patients is common in countries where drug prescription is not well regulated. Incorrectly, a patient may use antibiotics to alleviate a medical condition when the antibiotic is not necessary. For example, toothaches and sore throats are common and often ignored, or self-treated by patients when in fact an antibiotic is not necessary or has no effect at all. In some cases, a sore throat is often a sign of upper airway irritation from air pollution, smoking, drinking and talking, where the correct treatment is simply rest, symptomatic relief and to refrain from the hazardous environment. When other conditions exist, the symptom may be due to viral infection and insensitive bacterial infection in which case antibiotics are ineffective.

Therefore, It is desired to have a device with the following characteristics: 1. Performing visual examination and inspection of the oral cavity and upper airway in connection with common upper respiratory symptoms; 2. This device can be easily used by patient for self-examination, by a family member as well as by physicians; 3. Noninvasive with minimum noxious stimulation; 4. Most importantly, this device can be used even with the mouth closed. Furthermore, this device has to have memory, so serial examination and subsequent comparison can be made to monitor the progression of the symptoms and signs. The inventors recognize the problem, and take advantage of image sensing and other sensing technology to create a novel flexible oral scope. When inserted into one's mouth, this novel oral scope can capture images of upper airway including the mouth, teeth, tongue, tonsils, adenoid, and even the vocal cord while sensing the temperature. This will significantly extend one's ability to make self discovery of his or her own abnormalities and to make informed decisions on whether to seek early medical attention. This will also aid both patients and physicians to monitor the response to treatment early and objectively.

### SUMMARY OF THE DISCLOSURE

The disclosure presented here is directed to an instrument in which a person can use to inspect the mouth, in particular, the oral cavity and upper airway to ascertain if there is an abnormality which can be seen, that relates to a symptom that the person has, or the person's children or others may have. The instrument may be used in conjunction with a system including a display such as a monitor, smart phone, TV and other devices, along with data storage for retaining images and temperature information for serial exam and comparison.

The instrument can also be used with a system that in addition to display and data storage includes data processing, in particular with software for pattern recognition and/or pattern matching, thereby to give guidance for self diagnosis, and most importantly seeking medical attention with confidence. Additionally, the instrument may be effectively incorporated with a system which will upload images and temperature data into a website for storage, data processing, including pattern matching and/or recognition for review by a qualified physician for diagnosis, along with treatment options, including physician referrals in the area where the person lives.

The instrument itself may take several different forms or embodiments, which will be disclosed here, but the essential form and structure in its basic aspect, is as follows: An oral scope for facilitating visual examination and inspection of a person's oral cavity and upper airway includes an elongate, flexible arm having first and second ends, a temperature sensor is connected to the flexible arm adjacent the first end and an image sensor is mounted adjacent the second end in electrical communication with an image display device. A handle connected to the first end for enabling manual guiding and insertion of the flexible arm into the mouth along at least a portion of the top of the tongue to engage the temperature sensor therewith and to position the image sensor for viewing a selected area of the oral cavity of the upper airway. An illumination unit located adjacent the second end provides illumination for the image sensor. The image sensor, while operable for displaying a visual image of the oral cavity and upper airway, may include an infrared camera, and be a thermal image sensor of the tissue being specifically examined. The temperature sensor that engages the tongue will measure the body's temperature.

A person uses the oral scope by gripping the handle with the thumb, forefinger and if desired, the inserting it into the mouth, and guiding it along the tongue, to illuminate an area that the person believes may the situs of the tooth ache, sore throat, sore tonsils, gum inflammation and pain, whatever the case may be. The temperature sensor is located on the flexible arm adjacent its connection to the handle, so that when the flexible arm is inserted in the mouth, and the mouth is closed, the temperature sensor engages the top of the tongue, near the front of the tongue.

Prior to insertion of the flexible arm, it may be bent, upwardly, downwardly or to either side, in anticipation to where the person believes the problem may be. This is to position the image sensor for viewing a selected area of the oral cavity or upper airway. The image sensor communicates with a display, such as a monitor, and an image of the area can be seen, confirming or rejecting or perhaps inconclusive as to what the abnormality or problem is. Then the flexible arm possibly would be repositioned in the mouth to focus the image sensor on another area. Or the flexible arm could be removed, perhaps bent to a different position and angle, and reinserted in the mouth for visual examination and inspection.

There are other embodiments of the oral scope instrument that form part of the present disclosure. One embodiment incorporates an adjustment mechanism associated and cooperating with the flexible arm for enabling the second end of the flexible arm and the image sensor mounted thereon, to be selectively extended and retracted, relative to the handle. This is so the effective length of the scope may be made to extend further into the mouth, or made to extend less, important for the proper application of the instrument to the needs of children who have smaller oral cavities.

Another embodiment of the oral scope instrument contemplates that the image sensor may be selectively pivoted at a selected angle relative to the long axis of the flexible arm of the scope. To accomplish this functionality, an actuator is mounted on the handle operatively connected to the image sensor to selectively pivot and position the image sensor at a selected angle. This can be done by the person operating the oral scope when the instrument is in the mouth. This feature affords special benefit when the flexible arm is fully inserted; here the image sensor, if tilted upwardly can zoom in on the uvula and upper airway, if tilted downwardly can focus on the epiglottis, vestibular fold and adjacent anatomies.

A feature finding further utility is that the cross section of the flexible arm may be formed oval in shape and oriented so that when it is inserted in the mouth the long diameter of the oval is generally parallel to the top surface of the tongue enabling the flexible arm to engage the tongue and be at least partially guided by the tongue as the flexible arm is moved inwardly of the oral cavity.

### BRIEF DESCRIPTION OF THE DRAWINGS

FIG. 1 is a side view, with portions cut away, showing one embodiment of an oral scope having a flexible arm according to the present disclosure;

FIG. 2 is a side view, with portions cut away, showing another embodiment of an oral scope having a flexible arm and a housing and image sensor which are operable to be selectively pivoted relative to the long axis of the flexible arm according to the present disclosure;

FIG. 3 is an enlarged view of the end of the flexible arm and the housing and image sensor of the embodiment shown in FIG. 1;

FIG. 3A is an enlarged view of the end of the flexible arm and the housing and image sensor of the embodiment shown in FIG. 2;

FIG. 4 is a sagittal view of the anatomy of the throat and upper respiratory tract showing insertion of the flexible arm of the oral scope of FIG. 1 partially inserted in the mouth and engaging the tongue;

FIG. 5 is a side view, with portions cut away, showing another embodiment of an oral scope having an extendible-retractable flexible arm, with the flexible arm shown retracted in the handle in accordance with the present disclosure;

FIG. 5A is a side view, with portions cut away, showing the embodiment of FIG. 5 with the flexible arm shown fully extended;

FIG. 6 is a sagittal view of the anatomy of the throat and upper respiratory tract showing full extension and insertion of the flexible arm of the oral scope embodiment of FIGS. 5 and 5A;

FIGS. 7 and 7A are side views, with portions cut away, showing another embodiment of an oral scope having an extendible-retractable flexible arm combined a housing and image sensor which are operable to be selectively pivoted relative to the long axis of the flexible arm, according to the present disclosure;

FIG. 7B is an enlarged view of the end of the flexible arm and the housing and image sensor of the embodiment shown in FIGS. 7 and 7A;

FIG. 8 is a sagittal view of the anatomy of the throat and upper respiratory tract showing full extension and insertion of the flexible arm of the oral scope embodiment of FIGS. 7 and 7A showing the housing and image sensor operable to be selectively pivoted relative to the long axis of the flexible arm;

FIGS. 9 and 9A are side views, with portions cut away, showing another embodiment of an oral scope having a flexible arm and an actuator, mounted on the handle, including a rotatable pulley/axle assembly, operable to entrain cables to selectively pivot the housing and the image sensor relative to the long axis of the flexible arm.

FIGS. 9B and 9C are enlarged views, exploded and assembled, respectively, of the end of the flexible arm, showing the housing, image sensor, cooperating auxiliary housing sections and the cable connections;

FIGS. 10 and 10A are side views, with portions cut away, showing another embodiment of an oral scope having an extendible-retractable flexible arm combined with an actuator, mounted on the handle, including a rotatable pulley/axle assembly, operable to entrain cables to selectively pivot the housing and the image sensor relative to the long axis of the flexible arm; and

FIG. 11 is a flowchart illustrating numerous applications of the oral scope embodiments of the present disclosure in facilitating examination and investigation of the oral cavity and the upper airway.

### DETAILED DESCRIPTION

As described in more detail below, the present disclosure includes several embodiments of an oral scope used in a system for facilitating visual examination and inspection of the oral cavity and upper airway of a person's mouth. The oral scope can be used by a person for self-examination, to determine if a symptom in their oral cavity or upper airway is abnormal or pathological. The oral scope can also be used by an adult, such as a parent to examine the condition of children, to determine visually the location and condition of an area of the mouth or throat that a child appears to have symptoms of some abnormality. The oral scope, in its various embodiments, may utilize an image sensor, such as a still image camera, a video camera or a thermal image camera to capture or record an image, which may then be compared to normal anatomy, thereby to provide an initial diagnosis, and provide guidance for further treatment, if deemed necessary.

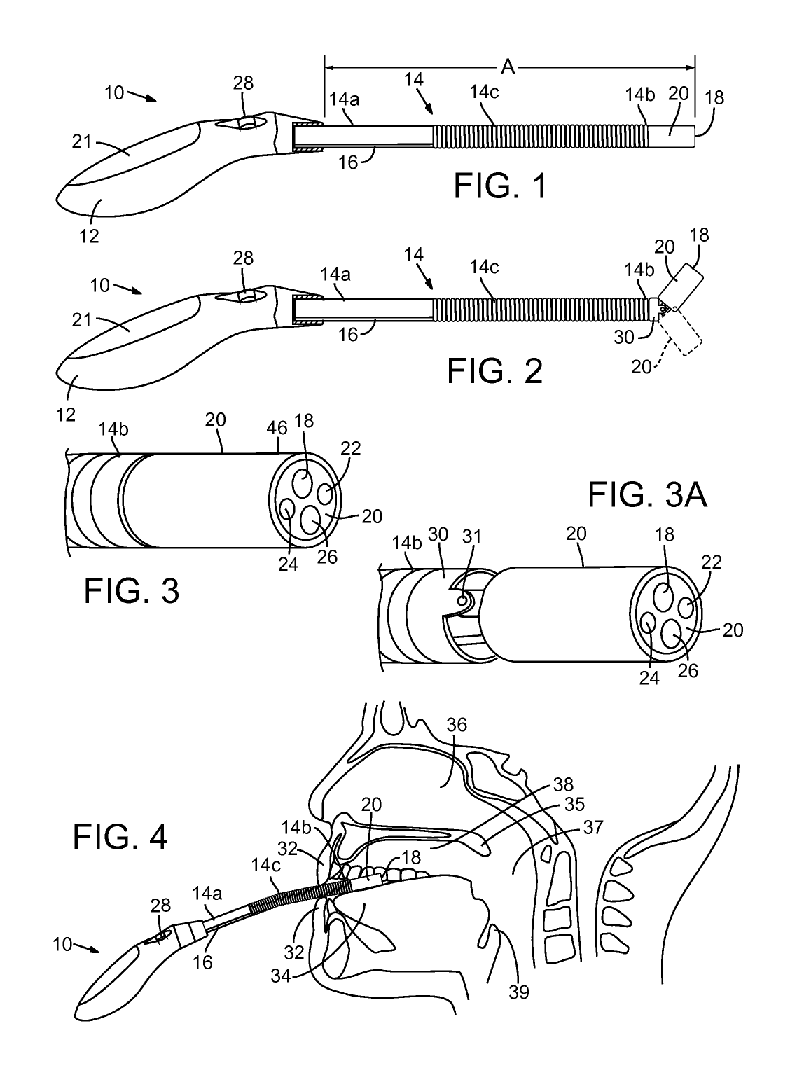

A first embodiment of an oral scope for facilitating visual examination and inspection of a person's oral cavity and upper airway is shown in FIG. 1, generally indicated at 10. The scope includes a handle 12, an elongate, flexible arm generally indicated at 14, and first and second ends 14a, 14b, respectively. First end 14a is a substantially rigid member, and may be fixed to handle 12, as shown. A temperature sensor, indicated at 16, is connected adjacent the first end, and may be a metallic piece or other material suitable for sensing temperature of a person, when inserted in the mouth, in a manner to be described.

A flexible section is indicated at 14c, and may be formed of corrugated plastic or metallic materials, and as shown can be of helix or spiral windings, the material is deformable, so that it can bent into a particular shape, and returned to its original shape. The flexible arm is deformable along its length for bending and orienting it to a preselected, non-permanent position. Additionally, the cross section of the flexible arm is oval and oriented so that when it is inserted in the mouth the long diameter of the oval is generally parallel to the top surface of the tongue enabling the flexible arm to engage the tongue and be at least partially guided by the top surface of tongue as the flexible arm is moved inwardly of the oral cavity.

An image sensor, generally indicated at 18, may be mounted in a housing 20, which is connected adjacent the second end. As shown in FIG. 3, housing 20 is rigidly connected to the second end of flexible arm 14. While not specifically shown in FIG. 1, or any of the figures of the other embodiments to be discussed, it will be understand that a processor for data storage, illumination, and display of images may be contained in the handle or first end 14a. The processor may be in electrical communication with image sensor 18, such as a still image camera, a video camera, a thermal camera and also with an image display device, which may be a screen such as indicated at 21 on handle 12, or a remote monitor, such as a computer screen, TV, cell phone, iPad or similar device.

Housing 20 contains image sensor 18, and an illumination unit for the image sensor may include LED's such as indicated at 22 and 24, operable by a control 28 on handle 12. Another port is shown at 26, adjacent to image sensor 18, and this may be for the administration of medicine. Or it could be adapted so that the image sensor operates under 3D. The length of flexible arm 14, shown at A in FIG. 1, may be in the range of 6-8 cm, a dimension sufficiently long for insertion into the mouth for visual examination. The cross section of the flexible scope may be oval, with the long diameter being in the range of 1-2 cm and the short diameter being in the range of 0.25-1 cm.

The use of the oral scope shown in FIG. 1 will now be described, with reference to FIG. 4 which is a sagittal view of the anatomy of the throat and upper respiratory tract. The lips are shown at 32, the tongue at 34, the nasal cavity at 36, the oral cavity at 38, the uvula at 35, the upper airway (oropharynx) at 37 and the epiglottis at 39. If, for example, a person experiences pain or discomfort or some other symptom in the mouth, they may want to see what the area looks like, it could be the gums, teeth, back of throat, or any of a number of other areas. The person may want to see if a selected area or anatomy is swollen, discolored, bleeding, or if a tooth, broken or cracked, or if the throat, having unusual redness, etc.

The flexible arm of the oral scope may first be bent, into any position that is believed will be directed to the area selected to be examined or investigated. The flexible arm may be bent up, down, to either side, midway or intermediate its length. This bending occurs when the scope is outside the mouth. Next handle 12 is gripped, with say, the thumb, middle, and forefinger, and the flexible arm is guided and inserted into the mouth, along at least a portion of the top of the tongue. As shown in FIG. 4, the mouth has been closed around the flexible arm, but it is not fully inserted, in fact the temperature sensor is not shown engaging the tongue. The flexible arm lies along the top of the tongue, and is illustrative of only one of many positions. The image sensor, for example a video camera, is activated for viewing and recording the anatomical features of the selected area of the oral cavity or the upper airway, and the processor is configured to store anatomical images and transmit images of the recorded anatomical features to the image display device, such as that indicated a 21, or any other such as a TV or monitor. While not specifically shown, the handle may be equipped with a hook up to an external display device.

Suffice it to say that this embodiment, no matter how far inserted would not likely be able to view deeper into the upper airway, to investigate farther down the throat, for example. However, the embodiment of FIG. 2 may accomplish this result. It includes the same basic structure and components as the embodiment of FIG. 1, but is enhanced by the addition of a rigid link or mount 30 interposed between the end of flexible arm 14 and housing 20. This construction enables housing 20 and image sensor 18 mounted within, to be pivoted relative to the flexible arm, either up or down, by an individual prior to the second end being inserted into the mouth. Mount 30 is connected to and aligned with the flexible arm for enabling the image sensor to be selectively pivoted and positioned at a selected angle relative to the long axis of the scope.

Thus, a person may initially pivot or tip the housing upwardly, to a selected angle, and insert the flexible arm. Depending on the length of insertion, better recordings of anatomies around the uvula, and upper airway may be accomplished. Of course the flexible tube itself may be bent, even to the side of the mouth. If the area selected for examination is the epiglottis, indicated at 39, or vestibular fold (not shown) better examination and focus will be accomplished by the housing and the image sensor being pivoted or tipped downwardly.

As mentioned previously, the oral scope of the present disclosure may be very useful for adults to examine children, for example parents may believe that a child is exhibiting symptoms that warrant investigation. But small children may feel uncomfortable, that they will choke, and therefore may well reject an oral scope dimensioned with what to them looks and feels like a long flexible arm. One solution is an embodiment provided with an adjustment mechanism, associated and cooperating with the flexible arm for enabling the second end of the flexible arm and the image sensor mounted thereon to be selectively extended and retracted, relative to the handle.

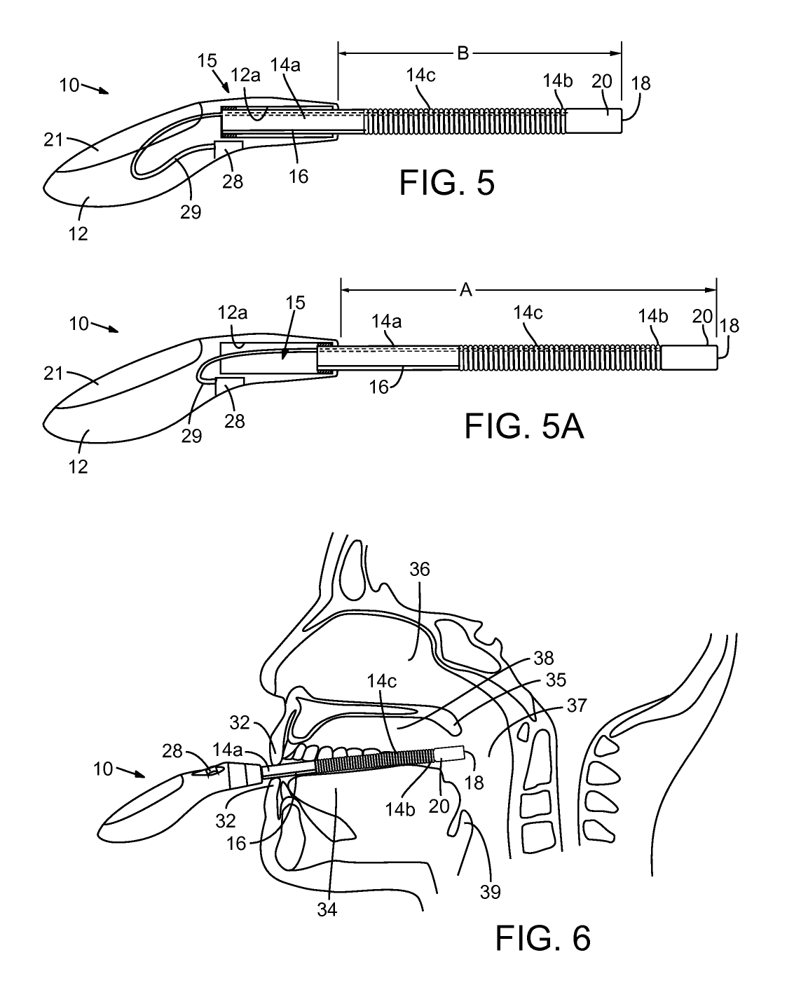

An example of this embodiment is disclosed in FIGS. 5 and 5A where an adjustment mechanism, generally indicated at 15, contemplates that handle 12 is provided with a bore 12a, dimensioned with a cross-section and length to receive a substantial portion of the flexible arm, when in the retracted position, as shown in FIG. 5. For example, if the maximum length of the flexible arm is say, 6 to 8 cm, as represented by the distance A in FIG. 5A, the bore may be dimensioned to with a length of 2 cm or thereabouts.

That would take off about ⅓ of the maximum length, and provide a much shorter effective length, as represented by B, more accommodating to children. The bore enables the effective inward and outward telescoping of the flexible arm, but the telescoping could be achieved at other locations along the length of the flexible arm. The flexible arm could be secured and released for extension or retraction within the bore in different ways. For example, the bore could be threaded for cooperating with threads on first end 14a, some threads are partially shown at 15. Or there could be a threaded nut provided adjacent the opening of the bore, for receiving first end 14a, during extension and retracted.

FIG. 6 illustrates oral scope 10 with the flexible arm fully extended and having been guided into position for viewing a selected area. For purposes of simplicity, and illustration, the flexible arm has not been bent, but the mouth is closed and lips 32 hold the oral scope so that temperature sensor 16 lays against and engages the top of the tongue, near the front of the tongue, where temperature readings are most accurate. The mouth is closed, preferably so as not to fog the lens of the image sensor, and the examination may proceed. It can be seen that housing 20 and image sensor 18 are extended for good viewing of the rear of the throat, but if it was desired to look higher up the back of the throat, or deeper downwardly, the arrangement shown has some limitations.

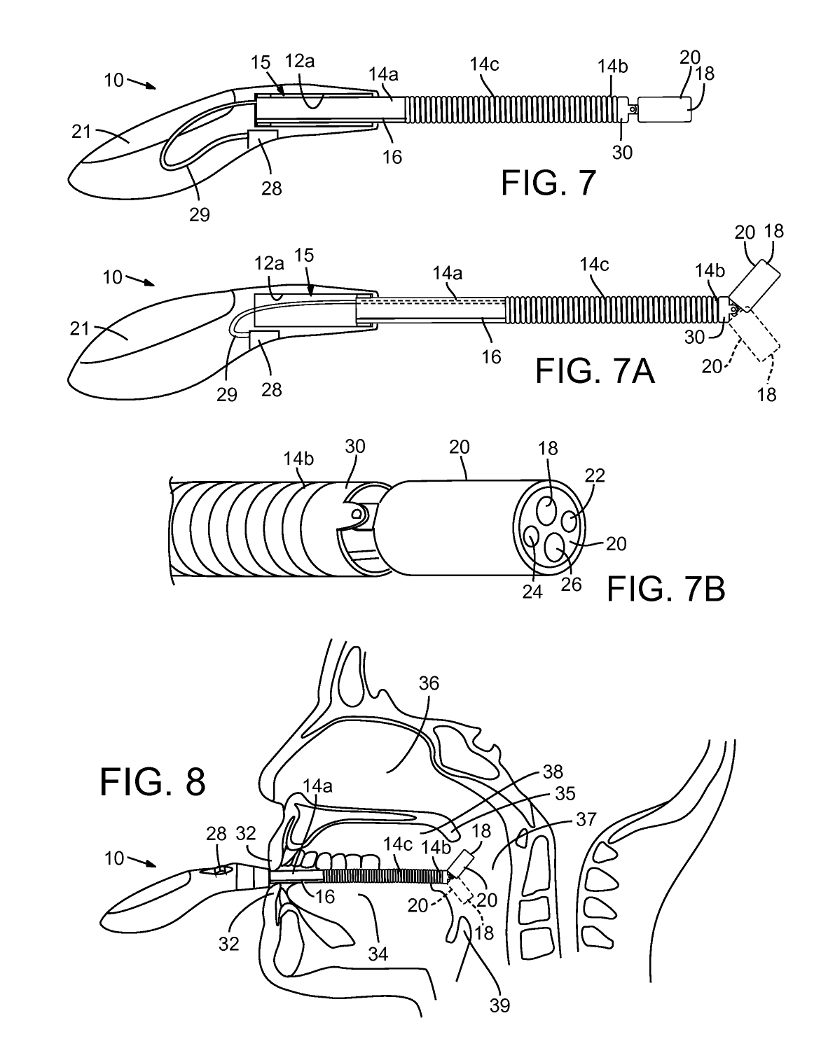

It would be advantageous to employ an actuator, as described previously, mounted on the handle operatively connected to the image sensor to selectively pivot and position the image sensor to a selected angle relative to the long axis of the scope. To that end, the combination of the actuator, and the adjustment mechanism, for extension and retraction of the flexible arm, are shown in the oral scope of FIGS. 7 and 7A. The FIG. 7 view shows flexible arm 7 fully retracted in bore 12a, and housing 20 and image sensor 18 are aligned with the long axis of the flexible arm. Now, it will be assumed that the area for examination does not require that the flexible arm be bent; but assume that it must be extended and the housing pivoted upwardly. Both of these functions are obtainable in the embodiment shown in FIGS. 7 and 7A. Initially before insertion in the mouth, housing 20 is manually pivoted upwardly (or downwardly) into a selected position.

The flexible arm, with the housing and the image sensor pivoted to the desired angle, is inserted into the mouth, which is then closed. This is the position shown in FIG. 8, and temperature sensor 16 engages the top surface of tongue 34. The image sensor is directed upwardly and a closer inspection of the upper airway can then be undertaken and recording of the anatomical features where symptoms may have arisen, or where visual inspection is sought.

The housing and image sensor could previously have been pivoted downwardly, prior to insertion into the mouth, so that the geometry would be shown by the dotted lines for the housing and image sensor, when inserted in the mouth, as shown in FIG. 8. It is to be remembered that extension and retraction may be selected. The flexible arm may be extended only partially outwardly from the handle; moreover the flexible arm may be bent. There are many possibilities, the oral scope is very universal in how it may have its shape, extension and image sensor positions selected, changed and oriented.

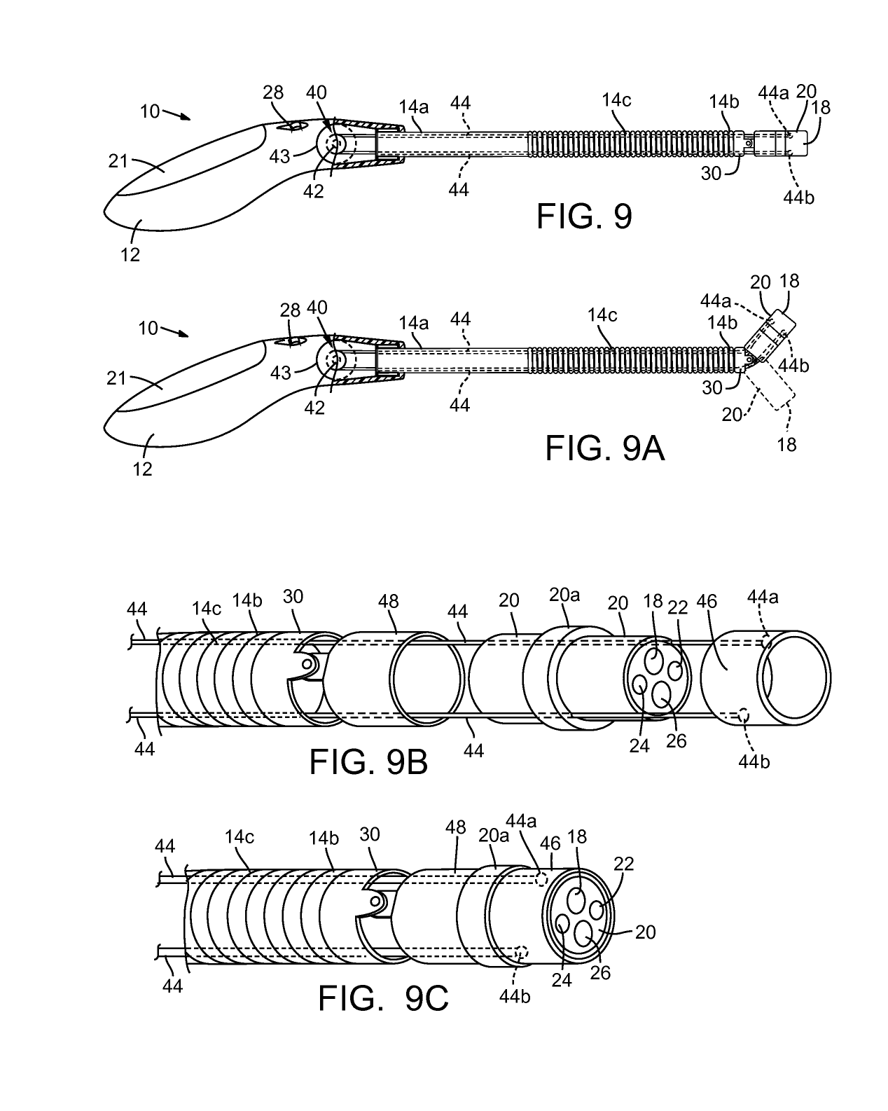

Which leads to yet a further embodiment where the actuator is remote, that is mounted on the handle and operable to selectively pivot and position the image sensor at a selected angle relative to the long axis of the flexible arm when inserted at least partially into the mouth. This enables the user to scan, using the pivot function, as desired, when the flexible arm is in situ, a very advantageous feature. The user can scan and simultaneously view on a display device what the image sensor is viewing. And then can reorient the angle of housing 20 and image sensor 18, either up or down, and in between. The structure that efficiently accomplishes all this is shown in FIGS. 9, 9A, 9B and 9C.

As shown in FIG. 9, an actuator assembly, generally at 40, is connected by elongate structure to housing 20, and is selectively operable to pivot the housing, and associated image sensor 18, either upwardly for viewing anatomies such as the uvula and the upper airway, or downwardly for viewing anatomies such as the epiglottis and vestibular fold. Actuator assembly 40 includes a rotatable pulley/axle assembly 42, wherein the pulley operates to entrain a cable 44 secured to an auxiliary housing section such as a cylinder 46 (see FIGS. 9A and 9B) at connection points 44a and 44b.

Cylinder 46 and a cooperating auxiliary housing section such as an aligned cylinder 48, shown in the exploded view of FIG. 9B, receive housing 20 and image sensor 18, as shown in the assembled view of FIG. 9C (a spacer is shown at 20a). A knob, button, or similar structure is shown at 43, and is actuated by the user for rotation, either clockwise or counterclockwise to rotate the pulley and thereby move cable 44 to tip or rotate the housing and image sensor either upwardly or downwardly, as shown in FIG. 9A. All this can of course be accomplished while the flexible arm is disposed in the mouth, either fully or partially.

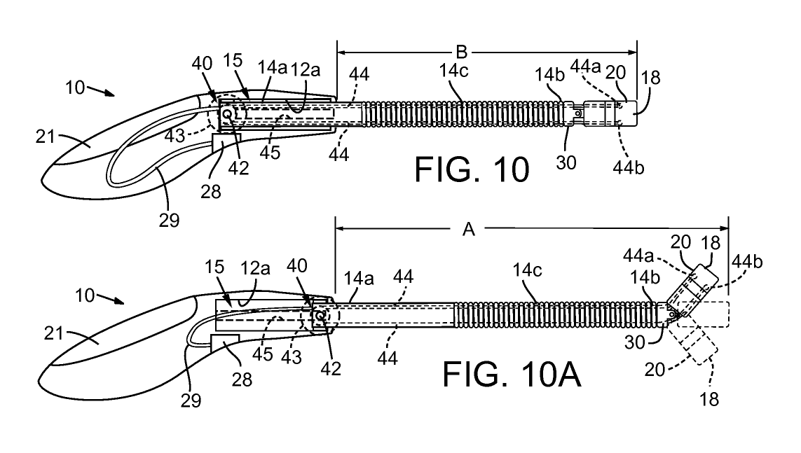

Further advantages to the oral scope can be appreciated in an embodiment which combines the functionality of the actuator assembly, as described above, and the adjustment mechanism, operable for selective extension/retraction of the flexible arm. As shown in FIGS. 9 and 10, these two functionalities are combined in oral scope 10, which includes actuator assembly 40 and an adjustment mechanism like that shown at 15 in FIGS. 5 and 5A. That adjustment mechanism utilizes a bore in the handle, and can be adapted as shown in FIGS. 9 and 10. There is included an elongate slot 45 in the wall of the handle, for the pulley/axle assembly to be shifted, as the flexible arm is moved, either as it is extended or retracted.

Most generally, the method of the present disclosure, for facilitating visual examination, inspection and analysis of a person's oral cavity and upper airway, includes the following steps: Providing an elongate, flexible arm having a temperature sensor adjacent one end thereof and an image sensor mounted adjacent the other end in electrical communication with an image display device, and illuminating the image sensor. The flexible arm is then manually guided and inserted into the mouth along at least a portion of the top of the tongue to engage the temperature sensor therewith and to position the image sensor for viewing and recording images of the anatomical features of a selected area of the oral cavity or the upper airway.

Images of the selected, viewed area of the oral cavity or upper airway are displayed on a monitor and temperature data obtained during the inspection are recorded and stored. Images anatomical features are displayed for pattern matching or recognition with the images of the anatomical features recorded by the image sensor for an initial analysis and diagnosis of the abnormality, or pathology, if any, represented by the recorded images. If desired, the images recorded and temperature data may be transmitted to the server of a medical provider for analysis and diagnosis.

### Method of Using the Oral Scope

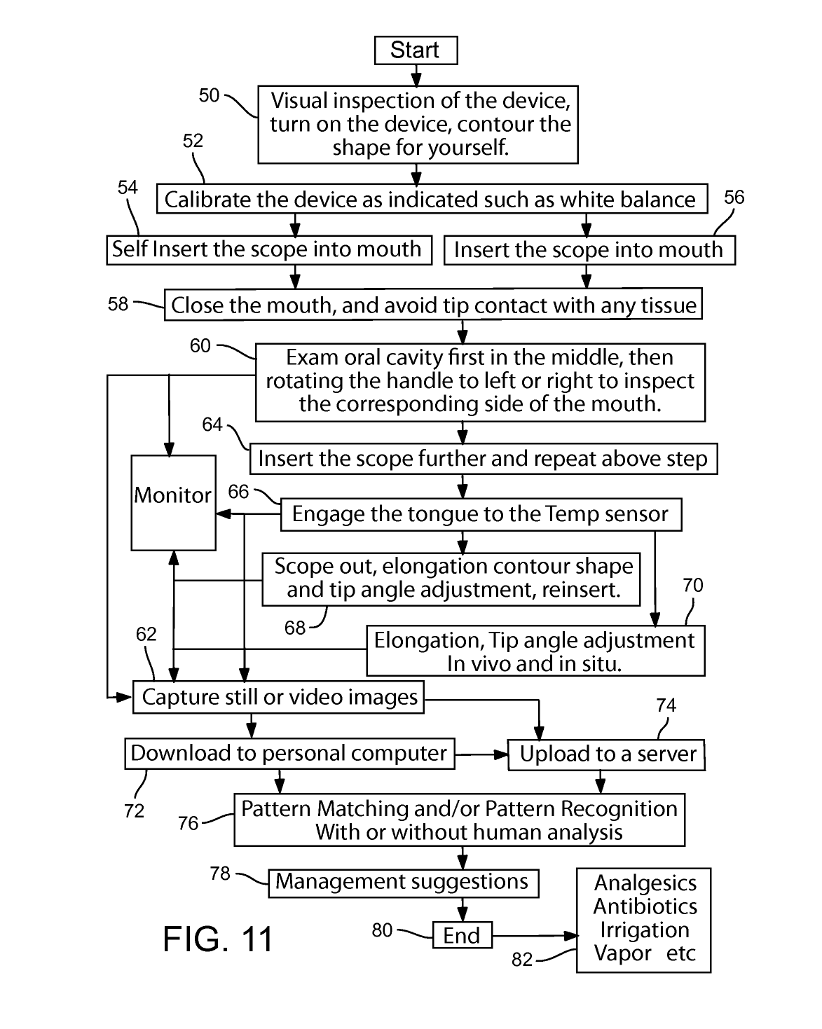

FIG. 11 lays out examples of a sequential overview of the method of using the oral scope of the present disclosure, in its various embodiments, for facilitating visual examination and inspection of a person's oral cavity and upper airway. Initially, at 50, a person inspects the oral scope, turns on the power, and contours the shape, as by bending the flexible arm or pivoting the housing and image sensor, if pivoting is a feature enabled by the particular embodiment. The device is then calibrated, as at 52, and inserted in the mouth, either by the person for self-examination at 54, or into the mouth of another, at 56. Next, at 58, the mouth is closed (in some cases open for short duration), and tip contact by the image a sensor with tissue is sought to be avoided.

As shown at 60, initial oral examination is conducted in the front or middle of the mouth, by rotating the handle to the left and right to inspect corresponding sides of the mouth, and the person can view still or video images on a monitor, and as shown at 62, these can be recorded or captured and downloaded to a personal computer as at 72 and or uploaded to a server as at 74. Or, after being downloaded to a personal computer as at 72, then uploaded to a server as at 74. Then as at 76 pattern matching and/or recognition with or without human analysis is an option, followed by management suggestions, as to treatment, depending on the condition, as at 78. This may be followed by treatment, as with for example, analgesics, antibiotics, irrigation, vapor or other modalities, as at 82.

If after initial oral exam at 60, more investigation is deemed warranted, the scope may be further inserted, as at 64, and the steps as just recited may be repeated. Next, if desired, as at 66, the temperature sensor may be engaged with the tongue, and an image of the temperature may be seen at the monitor. From here, the path may be followed to steps 62, 72, and/or 74 or 72 and then 74, and thenceforth to steps 76-82, as desired, or deemed necessary. However, after step 66, there are two other paths which may be followed. First, as shown at 68, the flexible arm may be withdrawn, and the flexible arm extended, and its contour modified or shaped, i.e., bent to the form deemed best for further investigation.

The housing and image sensor may tipped or pivoted, as well, relative to the long axis of the flexible arm, to establish the desired orientation, either up or down, of the image sensor. If however, the oral scope embodiment is that shown in FIGS. 10 and 10A, the step at 70 may be followed, whereby the flexible arm may be extended and the image sensor pivoted, in vivo and in situ, that is while in the mouth, and while the person views the monitor. Thereafter, whether path 68 or 70 is the applicable one, or the one followed, the remaining paths or steps 62-82 may be followed.

Importantly, note that the various steps or functions illustrated may be performed in the sequence illustrated, in parallel, or in some cases omitted. Likewise, the order of processing is not necessarily required to achieve the features and advantages of the example embodiments described herein, but is provided for ease of illustration and description. One or more of the illustrated steps or functions may be repeatedly performed depending on the particular strategy being used. Further, the described steps may graphically represent code to be programmed into the processor.

We claim:

1. An oral scope instrument for facilitating visual examination and inspection of a person's oral cavity and upper airway comprising:
an elongate, flexible arm having first and second ends;
a temperature sensor connected to the flexible arm adjacent the first end;
an image sensor mounted adjacent the second end;
a processor in electrical communication with the image sensor and an image display device;
an illumination unit located adjacent the second end for providing illumination for the image sensor; and
a handle connected to the first end for enabling manual guiding and insertion of the flexible arm into the mouth along at least a portion of the top of the tongue to engage the temperature sensor therewith and to position the image sensor for viewing and recording the anatomical features of a selected area of the oral cavity or the upper airway, wherein the processor is configured to store normal anatomical images and transmit images of the recorded anatomical features to the image display device.

2. The instrument as defined in claim 1 wherein the flexible arm is deformable along its length for bending and orienting it to a preselected, non-permanent position.

3. The instrument as defined in claim 2 wherein the image sensor is mounted to the flexible arm so that it may be pivoted relative to the flexible arm, prior to the second end being inserted in the mouth.

4. The instrument as defined in claim 3 wherein the second end includes a mount connected to and aligned with the flexible arm for enabling the image sensor to be selectively pivoted and positioned at a selected angle relative to the long axis of the scope.

5. The instrument as defined in claim 1 wherein the cross section of the flexible arm is oval and oriented so that when it is inserted in the mouth the long diameter of the oval is generally parallel to the top surface of the tongue enabling the flexible arm to engage the tongue and be at least partially guided by the tongue as the flexible arm is moved inwardly of the oral cavity.

6. An oral scope instrument for facilitating visual examination and inspection of a person's oral cavity and upper airway comprising:
an elongate, flexible arm having first and second ends;
a temperature sensor connected to the flexible arm adjacent the first end;
an image sensor mounted adjacent the second end in electrical communication with an image display device;
a processor in electrical communication with the image sensor and an image display device;
an illumination unit located adjacent the second end for providing illumination for the image sensor;
a handle connected to the first end for enabling manual guiding and insertion of the flexible arm into the mouth along at least a portion of the top of the tongue to engage the temperature sensor therewith and to position the image sensor for viewing and recording the anatomical features of a selected area of the oral cavity or the upper airway, wherein the processor is configured to store normal anatomical images and transmit images of the recorded anatomical features to the image display device; and
an adjustment mechanism associated and cooperating with the flexible arm for enabling the second end of the flexible arm and the image sensor mounted thereon to be selectively extended and retracted, relative to the handle.

7. The instrument as defined in claim 6 wherein the adjustment mechanism is mounted on the handle and receives the flexible arm, and enables the flexible arm to be selectively extended and retracted.

8. The instrument as defined in claim 7 wherein the flexible arm is deformable along its length for bending and orienting it to a preselected, non-permanent position.

9. The instrument as defined in claim 8 wherein the second end includes a mount connected to and aligned with the flexible arm for enabling the image sensor to be selectively pivoted and positioned at a selected angle relative to the long axis of the scope.

10. An oral scope instrument for facilitating visual examination and inspection of a person's oral cavity and upper airway comprising:
an elongate, flexible arm having first and second ends;
a temperature sensor connected to the flexible arm adjacent the first end;
an image sensor mounted adjacent the second end in electrical communication with an image display device;
a processor in electrical communication with the image sensor and an image display device;
an illumination unit located adjacent the second end for providing illumination for the image sensor;
a handle connected to the first end for enabling manual guiding and insertion of the flexible arm into the mouth along at least a portion of the top of the tongue to engage the temperature sensor therewith and to position the image sensor for viewing and recording the anatomical features of a selected area of the oral cavity or the upper airway, wherein the processor is configured to store normal anatomical images and transmit images of the recorded anatomical features to the image display device; and
an actuator mounted on the handle operatively connected to the image sensor to selectively pivot and position the image sensor at a selected angle relative to the long axis of the scope.
an illumination unit located adjacent the second end for providing illumination for the image sensor.

11. The instrument as defined in claim 10 wherein the image sensor is mounted in housing and the actuator is connected by elongate structure to the housing and is selectively operable to pivot the housing, and the associated image sensor, either upwardly for viewing the uvula and the upper airway, or downwardly for viewing the epiglottis and vestibular fold.

12. The instrument as defined in claim 11 wherein the elongate structure includes cable elements interconnecting the actuator to the housing, and the actuator is operable by a person's fingers to pivot the housing and the associated image sensor.

13. The instrument as defined in claim 12 wherein the flexible arm is deformable along its length for bending and orienting it to a preselected, non-permanent position.

14. The instrument as defined in claim 13 wherein the second end includes a mount connected to and aligned with the flexible arm for enabling the image sensor to be selectively pivoted and positioned at a selected angle relative to the long axis of the scope.

15. An oral scope instrument for facilitating visual examination and inspection of a person's oral cavity and upper airway comprising:
an elongate, flexible arm having first and second ends;
a temperature sensor connected to the flexible arm adjacent the first end;
an image sensor mounted adjacent the second end in electrical communication with an image display device;
a processor in electrical communication with the image sensor and an image display device;
an illumination unit located adjacent the second end for providing illumination for the image sensor;
a handle connected to the first end for enabling manual guiding and insertion of the flexible arm into the mouth along at least a portion of the top of the tongue to engage the temperature sensor therewith and to position the image sensor for viewing and recording the anatomical features of a selected area of the oral cavity or the upper airway, wherein the processor is configured to store normal anatomical images and transmit images of the recorded anatomical features to the image display device;
an adjustment mechanism associated and cooperating with the flexible arm for enabling the second end of the flexible arm and the image sensor mounted thereon, to be selectively extended and retracted, relative to the handle; and
an actuator mounted on the handle operatively connected to the image sensor to selectively pivot and position the image sensor at a selected angle relative to the long axis of the scope.

16. The instrument as defined in claim 15 wherein the image sensor is mounted in housing and the actuator is connected by elongate structure to the housing and is selectively operable to pivot the housing, and the associated image sensor, either upwardly for viewing the uvula and the upper airway, or downwardly for viewing the epiglottis and vestibular fold.

17. The instrument as defined in claim 16 wherein the elongate structure includes cable elements interconnecting the actuator to the housing, and the actuator is operable by a person's fingers to pivot the housing and the associated image sensor.

18. The instrument as defined in claim 17 wherein the flexible arm is deformable along its length for bending and orienting it to a preselected, non-permanent position.

19. The instrument as defined in claim 18 wherein the second end includes a mount connected to and aligned with the flexible arm for enabling the image sensor to be selectively pivoted and positioned at a selected angle relative to the long axis of the scope.

20. The instrument as defined in claim 19 wherein the adjustment mechanism is mounted on the handle and receives the flexible arm, and enables the flexible arm to be selectively extended and retracted.

21. The instrument as defined in claim 20 wherein the second end includes a mount connected to and aligned with the flexible arm for enabling the image sensor to be selectively pivoted and positioned at a selected angle relative to the long axis of the scope.

22. The instrument as defined in claim 21 wherein the cross section of the flexible arm is oval and oriented so that when it is inserted in the mouth the long diameter of the oval is generally parallel to the top surface of the tongue enabling the flexible arm to engage the tongue and be at least partially guided by the tongue as the flexible arm is moved inwardly of the oral cavity.

23. The instrument as defined in claim 15 wherein a delivery port is provided on the flexible arm in communication with a delivery port on the handle for administering one of antibiotics, analgesics and cleaning liquid and to collect local tissues or exudates on the throat or tonsils for further diagnosis.

24. A method for facilitating visual examination, inspection and analysis of a person's oral cavity and upper airway comprising:
providing an elongate, flexible arm having a temperature sensor adjacent one end thereof and an image sensor mounted adjacent the other end in electrical communication with an image display device;
illuminating the image sensor;
manually guiding and inserting the flexible arm into the mouth along at least a portion of the top of the tongue to engage the temperature sensor therewith and to position the image sensor for viewing and recording images of the anatomical features of a selected area of the oral cavity or the upper airway; and
displaying images of the selected, viewed area of the oral cavity or upper airway on a monitor and recording and storing temperature data obtained during the inspection.

25. The method of claim 24 further including the step of storing images of normal anatomical features and displaying them for pattern matching or recognition with the images of the anatomical features recorded by the image sensor for an initial analysis and diagnosis of the abnormality, or pathology, if any, represented by the recorded images.

26. The method of claim 25 further including the step of displaying temperature data, stored images of normal anatomical features and images recorded by the image sensor on a T.V. screen, personal computer monitor or cell phone screen.

27. The method of claim 26 further including transmitting the images recorded and temperature data to the server of a medical provider for analysis and diagnosis.
